(19) United States
(12) Patent Application Publication
(10) Pub. No.: US 2023/0372676 A1
(43) Pub. Date: Nov. 23, 2023

(54) CATHETER FIXING DEVICE

(71) Applicant: TERUMO KABUSHIKI KAISHA, Tokyo (JP)

(72) Inventor: Shinichi Mizuno, Nakakoma-gun (JP)

(73) Assignee: TERUMO KABUSHIKI KAISHA, Tokyo (JP)

(21) Appl. No.: 18/365,800

(22) Filed: Aug. 4, 2023

Related U.S. Application Data
(63) Continuation of application No. PCT/JP2022/008886, filed on Mar. 2, 2022.

(30) Foreign Application Priority Data
Mar. 3, 2021 (JP) 2021-033755

(51) Int. Cl. A61M 25/02

(52) U.S. Cl. CPC A61M 25/02; A61M 2025/028; A61M 2025/0266

(57) ABSTRACT

A catheter fixing device includes a skin fixation part configured to come into contact with skin, a hub fixation part configured to retain a hub, and a movement mechanism provided between the skin fixation part and the hub fixation part and interlocking the hub fixation part with the skin fixation part such that the hub fixation part and the skin fixation part are movable relative to each other.

### CROSS-REFERENCE TO RELATED APPLICATIONS

This is a bypass continuation of PCT Application No. PCT/JP2022/008886, filed on Mar. 2, 2022, which claims priority to Japanese application number 2021-033755, filed on Mar. 3, 2021. The contents of these application are incorporated herein by reference in their entireties.

### BACKGROUND

### 1. Field of the Invention

The present disclosure relates to a catheter fixing device that fixes a catheter on skin.

### 2. Description of Related Art

In the related art, in order to prevent displacement of a catheter during indwell, a method of fixing the catheter with an adhesive tape or a method of using a catheter fixing device fixed on skin with a double-sided adhesive tape or the like is adopted (for example, JP2013-13446A).

However, in the related art, a fixing position of the catheter fixing device and a position of a puncture site of a blood vessel are changed due to body movement of a patient, and a distal end of a catheter may come out from the blood vessel.

### SUMMARY

In view of the above, an object of an embodiment is to provide a catheter fixing device capable of preventing a distal end of a catheter from coming out from a blood vessel even when skin moves due to body movement.

One aspect of the present disclosure is a catheter fixing device including: a skin fixation part configured to come into contact with skin; a hub fixation part configured to retain a catheter hub; and a movement mechanism provided between the skin fixation part and the hub fixation part and interlocking the hub fixation part with the skin fixation part to be movable relative to each other.

According to the catheter fixing device of the above aspect, it is possible to prevent a distal end of a catheter from coming out from a blood vessel even when the skin moves due to body movement.

### BRIEF DESCRIPTION OF THE DRAWINGS

FIG. 1 is a plan view of a catheter fixing device according to a first embodiment;

FIG. 2A is a cross-sectional view taken along a line IIA-IIA shown in FIG. 1, and FIG. 2B is a cross-sectional view taken along a line IIB-IIB shown in FIG. 1;

FIG. 3A is a plan view of a skin fixation part shown in FIG. 1, and FIG. 3B is an enlarged cross-sectional view of the vicinity of a sphere shown in FIG. 3A;

FIG. 4 is a diagram illustrating a use state of the catheter fixing device shown in FIG. 1;

FIG. 5 is a plan view of a catheter fixing device according to a second embodiment;

FIG. 6A is a side view of the catheter fixing device shown in FIG. 5, and FIG. 6B is a cross-sectional view taken along a line VIB-VIB shown in FIG. 5;

FIG. 7 is a plan view of a catheter fixing device according to a third embodiment;

FIG. 8A is a side view of the catheter fixing device shown in FIG. 7, and FIG. 8B is a cross-sectional view taken along a line VIIIB-VIIIB shown in FIG. 7;

FIG. 9 is a plan view of a catheter fixing device according to a fourth embodiment;

FIG. 10A is a side view of the catheter fixing device shown in FIG. 9, and FIG. 10B is a cross-sectional view taken along a line XB-XB shown in FIG. 9;

FIG. 11A is a plan view of a catheter fixing device according to a fifth embodiment, and FIG. 11B is a plan view of a skin fixation part shown in FIG. 11A; and

FIG. 12A is a cross-sectional view taken along a line XIIA-XIIA shown in FIG. 11A, and FIG. 12B is a cross-sectional view taken along a line XIIB-XIIB shown in FIG. 11A.

### DESCRIPTION OF EMBODIMENTS

Hereinafter, preferred embodiments of a catheter fixing device will be described in detail with reference to the accompanying drawings.

### First Embodiment

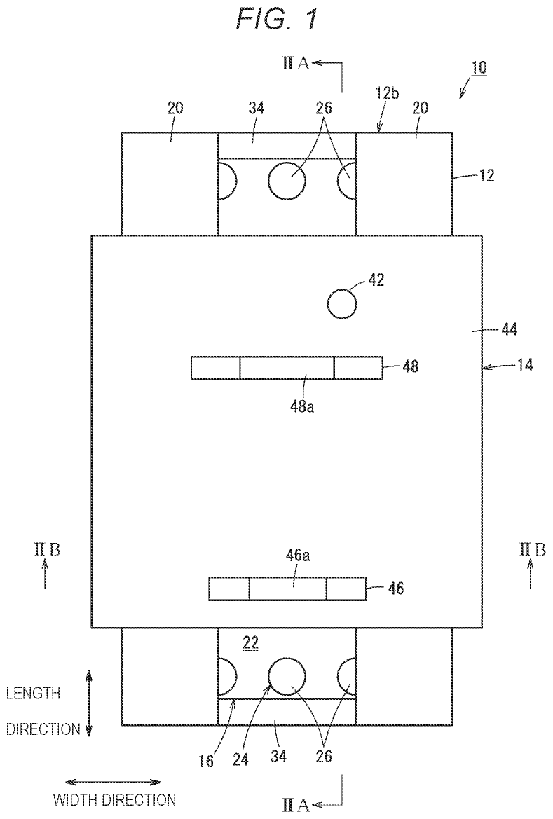

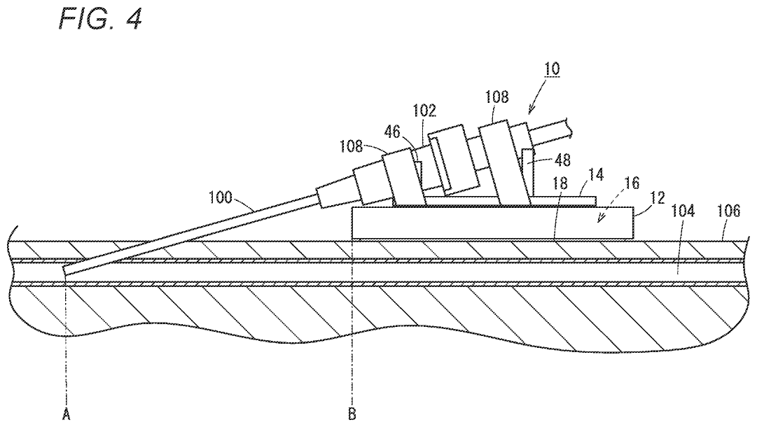

As shown in FIG. 1, a catheter fixing device 10 according to the present embodiment includes a skin fixation part 12 that comes into contact with skin 106 (see FIG. 4), a hub fixation part 14 that retains a hub 102 (see FIG. 4), and a movement mechanism 16 that is provided between the skin fixation part 12 and the hub fixation part 14 and interlocks the hub fixation part 14 with the skin fixation part 12 to be movable relative to each other.

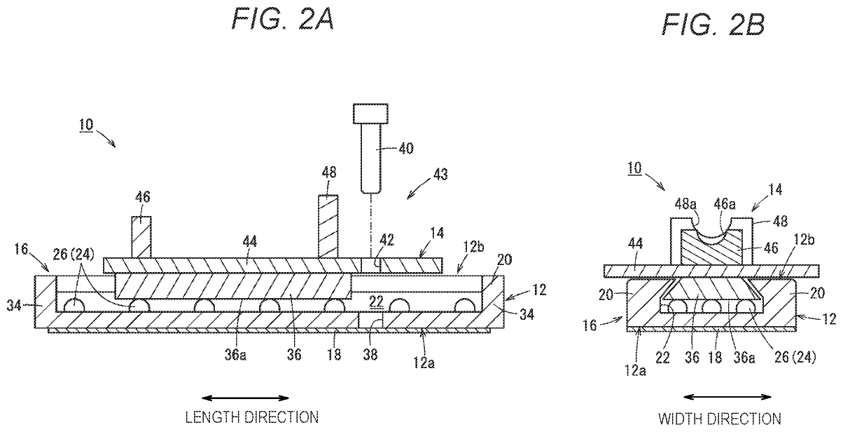

As shown in FIG. 2A, the skin fixation part 12 is formed in a thin plate shape, and has a flat fixing surface 12a on a side on which the skin fixation part 12 is attached to the skin 106. The fixing surface 12a is provided with an adhesive portion 18 made of an adhesive material. The skin fixation part 12 is fixed on the skin 106 by adhering an exposed surface of the adhesive portion 18 to the skin 106.

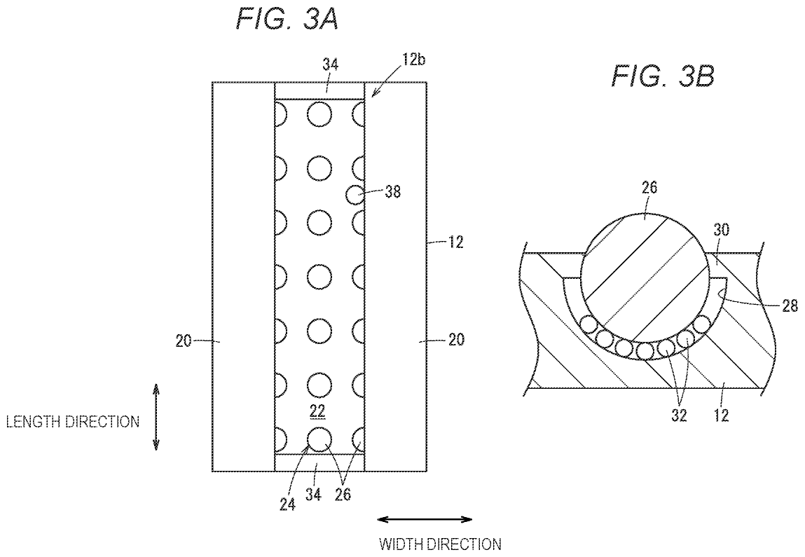

As shown in FIG. 3A, the skin fixation part 12 is formed in a rectangular shape in plan view. A length direction of the skin fixation part 12 is the same direction as an axis of a catheter 100 (see FIG. 4) described below. A pair of guide rails 20 are provided on an upper surface 12b (a surface opposite to the fixing surface 12a) of the skin fixation part 12. The guide rails 20 are provided on both side portions of the skin fixation part 12 in a width direction. The guide rails 20 extend in the length direction of the skin fixation part 12. A guide groove 22 extending at a constant width in the length direction of the skin fixation part 12 is formed between the pair of guide rails 20. As shown in FIG. 2B, a cross-sectional shape of the guide groove 22 formed between the guide rails 20 is narrower on an upper end side and wider on a lower side.

As shown in FIG. 3A, a bottom portion of the guide groove 22 is provided with a free ball bearing 24 constituting a part of the movement mechanism 16. The free ball bearing 24 includes a plurality of rotatable spheres 26. As shown in FIG. 3B, each sphere 26 is housed in a housing hole 28 provided in the bottom portion of the guide groove 22. The sphere 26 is retained by a retaining part 30. Small-diameter bearing balls 32 are disposed between the sphere 26 and the housing hole 28, and the bearing balls 32 rotatably support the sphere 26. The free ball bearing 24 includes the plurality of spheres 26, and thus supports the hub fixation part 14 to be displaceable in an in-plane direction of the skin fixation part 12 (fixing surface 12a).

As shown in FIG. 3A, stoppers 34 are provided respectively on one end and the other end in the length direction of the upper surface 12b of the skin fixation part 12. The stoppers 34 restrict a movement range of the hub fixation part 14 shown in FIG. 2A in the length direction of a slider part 36, and prevents the hub fixation part 14 from falling out from the guide groove 22.

A first lock hole 38 for fixing the hub fixation part 14 to the skin fixation part 12 is provided in a predetermined position of the guide groove 22 of the skin fixation part 12. As shown in FIG. 2A, the first lock hole 38 penetrates the skin fixation part 12 in a thickness direction, and has an inner diameter at which a distal end of a lock pin 40 can be inserted thereto. The first lock hole 38 is provided at a position overlapping a second lock hole 42 of the hub fixation part 14. The lock pin 40 is inserted into the first lock hole 38 and the second lock hole 42 to fix the hub fixation part 14 and the skin fixation part 12. The first lock hole 38, the second lock hole 42, and the lock pin 40 constitute a lock mechanism 43 according to the present embodiment.

As shown in FIG. 1, the hub fixation part 14 has a plate-shaped top plate portion 44, and the second lock hole 42 is formed in a predetermined portion of the top plate portion 44. In addition, provided on the top plate portion 44 are a first retaining protrusion 46 and a second retaining protrusion 48 that retain the hub 102 (which here and hereinafter refers to a member including a catheter hub and a lock connector fixed to a proximal end of the catheter hub) of the catheter 100. The first retaining protrusion 46 and the second retaining protrusion 48 are disposed at an interval in an extending direction of the guide groove 22.

As shown in FIG. 2B, the first retaining protrusion 46 is a member that retains a distal end side of the hub 102, and has an arc-shaped concave portion 46a. The second retaining protrusion 48 is a member that retains a proximal end side of the hub 102, and has an arc-shaped concave portion 48a having an inner diameter larger than that of the concave portion 46a. The concave portion 48a is provided above the concave portion 46a. Therefore, the hub fixation part 14 retains the hub 102 such that a distal end of the hub 102 is inclined downward.

As shown in FIGS. 2A and 2B, on a lower surface side of the hub fixation part 14, the slider part 36 is formed in a tapered shape that widens downward, and is not separated upward due to the guide groove 22 having a narrow upper end. A lower end portion of the slider part 36 is a flat surface 36a. The flat surface 36a contacts the plurality of spheres 26 of the free ball bearing 24. The slider part 36 is slidably retained by the spheres 26 in any direction along a plane of the skin fixation part 12. That is, the slider part 36 is also displaceable within a range of a gap between the guide groove 22 and the slider part 36 in the width direction.

The slider part 36 is also displaceable in the extending direction (length direction) of the guide groove 22 by being guided by the guide rails 20. A movement range of the slider part 36 in the extending direction of the guide groove 22 is restricted by the stoppers 34. In addition, a width of the slider part 36 is smaller than a width of the guide groove 22, and the gap is formed between the slider part 36 and the guide groove 22. The hub fixation part 14 is also displaceable in the width direction within a range of the gap between the slider part 36 and the guide groove 22. In the catheter fixing device 10 according to the present embodiment, the slider part 36 is displaceable farther in the length direction than in the width direction.

The movement mechanism 16 according to the present embodiment includes the pair of guide rails 20 forming the guide groove 22, the slider part 36 housed in the guide groove 22, and the free ball bearing 24 slidably supporting the slider part 36.

The catheter fixing device 10 according to the present embodiment is configured as described above, and an operation thereof will be described below.

As shown in FIG. 4, the catheter fixing device 10 is disposed under the hub 102 of the catheter 100 that is punctured into a blood vessel 104 of a patient. The skin fixation part 12 of the catheter fixing device 10 adheres to the skin 106 via the adhesive portion 18. The hub 102 of the catheter 100 is fixed to the hub fixation part 14 of the catheter fixing device 10. The hub 102 is retained in the concave portion 46a of the first retaining protrusion 46 and the concave portion 48a of the second retaining protrusion 48, and is retained in an inclined state such that the distal end side approaches the skin fixation part 12. Further, the hub 102 is fixed to the hub fixation part 14 by an adhesive member 108 such as a tape.

In an initial state of the catheter fixing device 10, the skin fixation part 12 and the hub fixation part 14 are fixed by the lock pin 40 (see FIG. 2A). After the hub 102 is fixed to the hub fixation part 14, the lock pin 40 is removed by a user such as a doctor or a nurse. Accordingly, in the catheter fixing device 10, the hub fixation part 14 retaining the hub 102 and the skin fixation part 12 fixed to the skin 106 are displaceable to each other through the movement mechanism 16.

When a puncture position A of the catheter 100 and a fixing position B of the skin fixation part 12 are changed by body movement of the patient, the hub fixation part 14 is displaced to maintain a position of the puncture position A. Therefore, according to the catheter fixing device 10 of the present embodiment, even when the body movement occurs, it is possible to prevent the distal end of the catheter 100 from coming off the blood vessel 104 (puncture position A).

### Second Embodiment

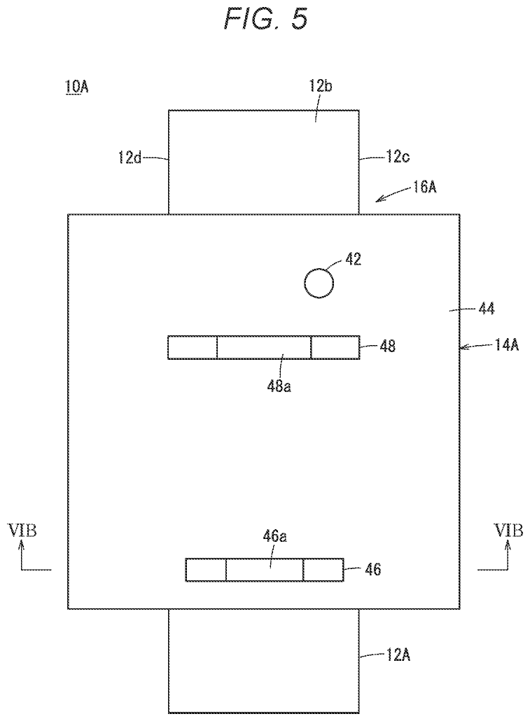

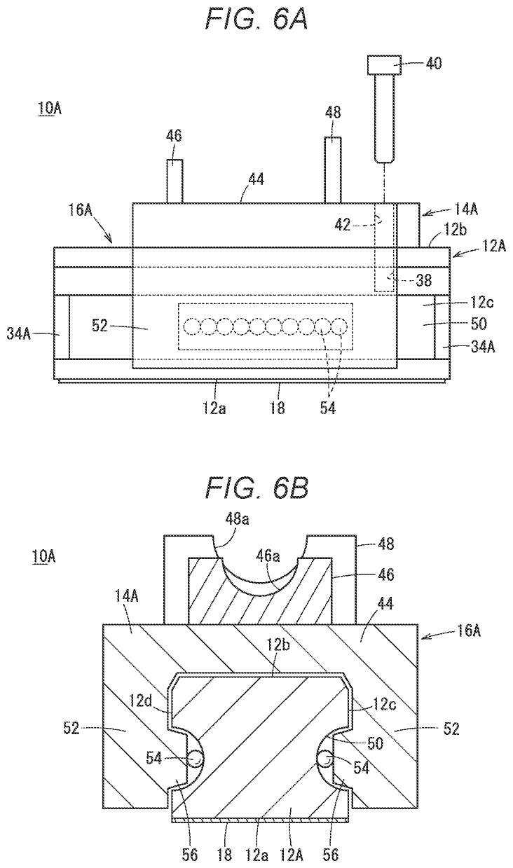

Hereinafter, a catheter fixing device 10A according to the present embodiment will be described with reference to FIGS. 5 to 6B. In the catheter fixing device 10A, the same components as those of the catheter fixing device 10 shown in FIG. 1 are denoted by the same reference numerals, and a detailed description thereof is omitted.

As shown in FIG. 5, the catheter fixing device 10A according to the present embodiment includes a skin fixation part 12A, a hub fixation part 14A, and a movement mechanism 16A. The skin fixation part 12A is formed in a rectangular shape in plan view. The upper surface 12b of the skin fixation part 12A is formed as a flat plane. As shown in FIG. 6A, a guide groove is formed in a lateral portion 12c of the skin fixation part 12A in a width direction. The guide groove 50 extends in a length direction of the skin fixation part 12A. As shown in FIG. 6B, the guide groove 50 is formed as an arc-shaped concave portion. As shown in FIG. 6A, stoppers 34A are provided on both ends of the guide groove 50. The guide groove 50 is also formed in a side portion 12d (see FIG. 5) on an opposite side of the skin fixation part 12A.

As shown in FIG. 5, the hub fixation part 14A includes the top plate portion 44 disposed on the upper surface 12b of the skin fixation part 12A. The first retaining protrusion 46 and the second retaining protrusion 48 are provided on the top plate portion 44. As shown in FIG. 6B, side wall parts 52 extending to cover outer sides of the side portions 12c and 12d of the skin fixation part 12 are formed on side portions in a width direction of the top plate portion 44, respectively. The side wall parts 52 cover the guide grooves 50 of the side portions 12c and 12d. A plurality of balls 54 and retaining parts 56 for retaining the balls 54 are provided at a part of the side wall parts 52 corresponding to the guide grooves 50, and the side wall parts 52 are in contact with the guide grooves 50 via the balls 54. In this way, the hub fixation part 14A and the skin fixation part 12A constitute the movement mechanism 16A including a linear guide (linear movement member).

As described above, in the catheter fixing device 10A according to the present embodiment, because the movement mechanism 16A can move the hub fixation part 14A along the extending direction of the catheter 100 (see FIG. 4), it is possible to effectively prevent the catheter 100 from falling off due to the body movement.

### Third Embodiment

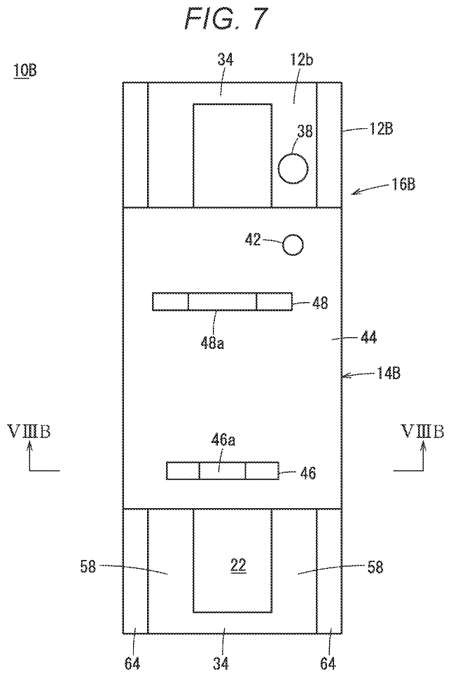

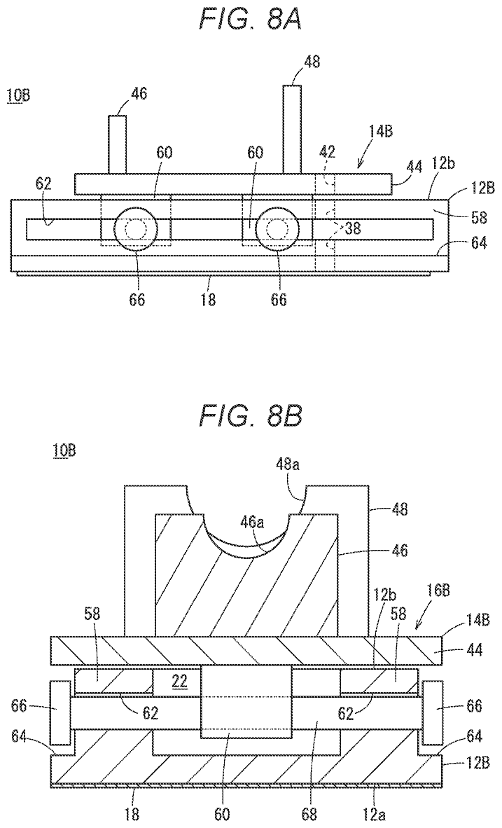

Hereinafter, a catheter fixing device 10B according to the present embodiment will be described with reference to FIGS. 7 to 8B. In the catheter fixing device 10B, the same components as those of the catheter fixing device 10 shown in FIG. 1 are denoted by the same reference numerals, and detailed description thereof is omitted.

As shown in FIG. 7, the catheter fixing device 10B according to the present embodiment includes a skin fixation part 12B, a hub fixation part 14B, and a movement mechanism 16B. The skin fixation part 12B is formed in a rectangular shape in plan view. On the upper surface 12b of the skin fixation part 12B, wall-like guide walls 58 are respectively provided on both side portions in a width direction. The pair of guide walls 58 extend from one end to the other end in a length direction of the skin fixation part 12B.

As shown in FIG. 8A, the guide walls 58 are each formed with a slide hole 62 penetrating the guide walls 58 in the width direction. The slide hole 62 extends along the length direction of the skin fixation part 12B.

As shown in FIG. 7, the guide groove 22 is formed between the pair of guide walls 58. As shown in FIG. 8B, the guide groove 22 houses, movably in the extending direction of the guide groove 22, bearing parts 60 constituting a part of the movement mechanism 16B. The stoppers 34 are provided on one end and the other end of the guide groove 22 to restrict a movement range of the bearing parts 60. As shown in FIG. 7, wheel housing grooves 64 for housing wheels 66 described below are formed outside the guide walls 58.

As shown in FIG. 7, the hub fixation part 14B includes the top plate portion 44 disposed above the guide walls 58 of the skin fixation part 12B. The first retaining protrusion 46 and the second retaining protrusion 48 are provided on the top plate portion 44. As shown in FIGS. 8A and 8B, the bearing parts 60, an axle 68, and the wheels 66 are provided on a lower surface (a surface on a skin fixation part 12B side) of the top plate portion 44. The pair of bearing parts 60 are spaced apart from each other in the length direction of the skin fixation part 12B. The bearing parts 60 rotatably support the cylindrical axle 68 extending in the width direction.

As shown in FIG. 8B, the axle 68 penetrates the slide holes 62 of the guide walls 58 in the width direction. The axle 68 can move while rolling in the slide holes 62. The wheels 66 are provided on both ends of the axle 68 in the width direction, respectively. The wheels 66 are rotatable integrally with the axle 68. The wheels 66 prevent the axle 68 from moving in the width direction.

Accordingly, the guide walls 58 each having the slide hole 62, the bearing parts 60, the axle 68, and the wheels 66 constitute the movement mechanism 16B according to the present embodiment having a wheel structure. In the catheter fixing device 10B according to the present embodiment, the hub fixation part 14B is interlocked with the skin fixation part 12B by the movement mechanism 16B to be movable relative to each other.

### Fourth Embodiment

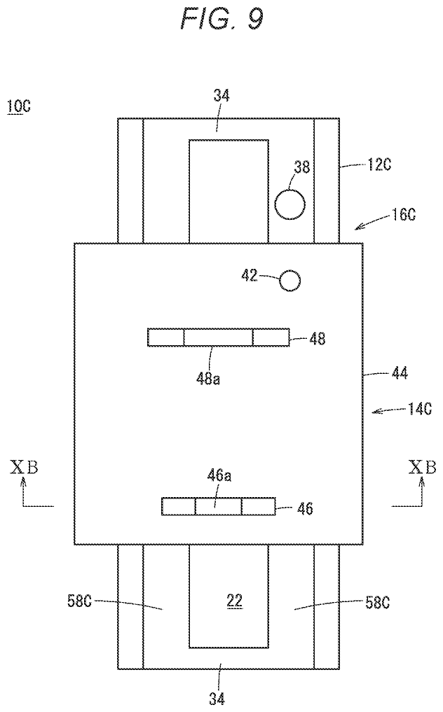

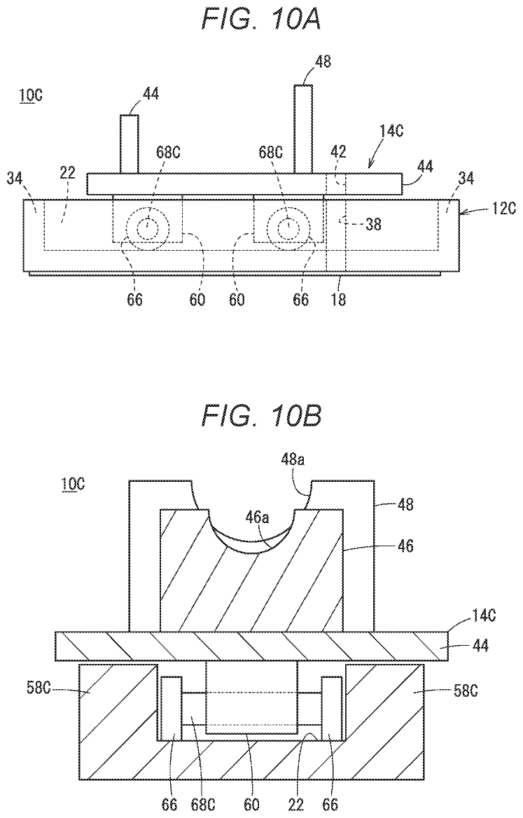

Hereinafter, a catheter fixing device 10C according to the present embodiment will be described with reference to FIGS. 9 to 10B. In the catheter fixing device 10C, the same components as those of the catheter fixing device 10B shown in FIG. 7 are denoted by the same reference numerals, and detailed description thereof is omitted.

As shown in FIG. 9, the catheter fixing device 10C includes a skin fixation part 12C, a hub fixation part 14C, and a movement mechanism 16C. In the catheter fixing device 10C according to the present embodiment, similarly to the catheter fixing device 10B shown in FIG. 7, the hub fixation part 14C and the skin fixation part 12C are interlocked by the movement mechanism 16C having a wheel structure to be movable relative to each other.

The skin fixation part 12C is provided with wall-like guide walls 58C on both side portions in a width direction. The pair of guide walls 58C extend from one end to the other end in a length direction of the skin fixation part 12C. The guide groove 22 restricting the movement range of the movement mechanism 16C is formed between the guide walls 58C.

As shown in FIG. 10A, the pair of bearing parts 60 are provided on a lower side of the hub fixation part 14C, and axles 68C and the wheels 66 are provided in the bearing parts 60, respectively. As shown in FIG. 10B, the axle 68C has a length that fits in the guide groove 22, and the axle 68C is housed in the guide groove 22 together with the wheels 66 on both ends. In the present embodiment, the wheels 66 are attached to a bottom portion of the guide groove 22 and are movable in the length direction of the skin fixation part 12C along the guide groove 22.

As described above, the catheter fixing device 10C according to the present embodiment also achieves the same effects as those of the catheter fixing device 10B shown in FIG. 7.

### Fifth Embodiment

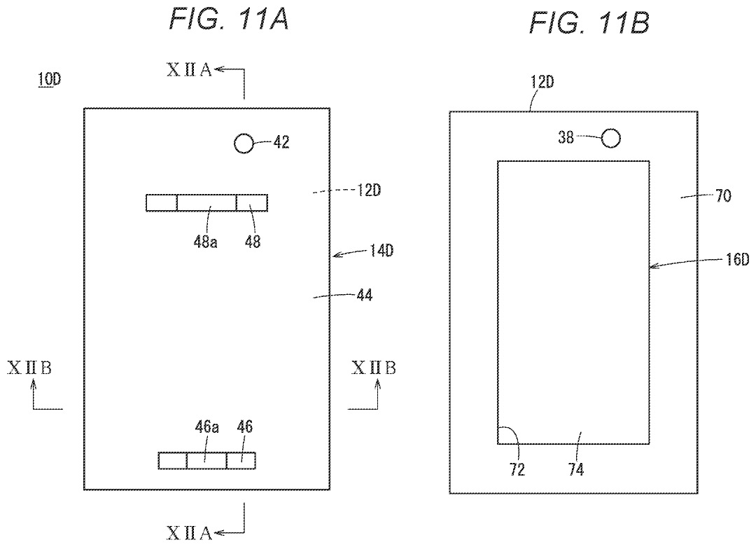

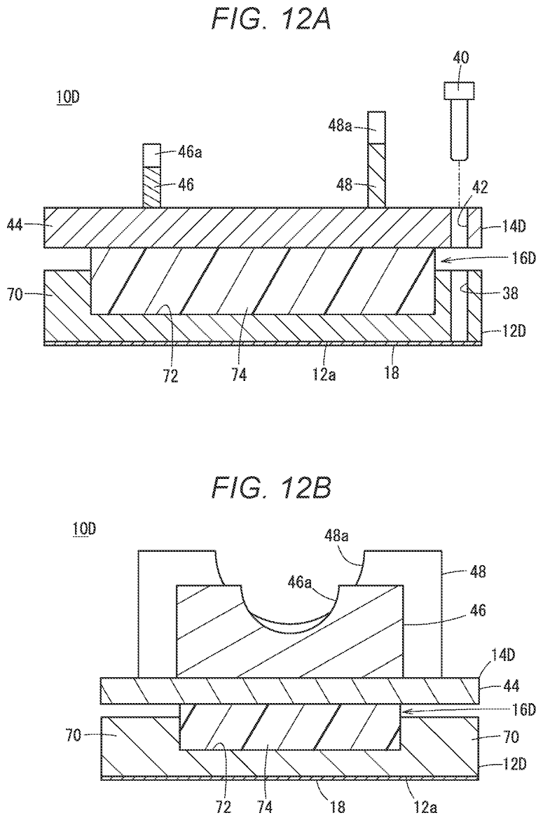

Hereinafter, a catheter fixing device 10D according to the present embodiment will be described with reference to FIGS. 11A to 12B. In the catheter fixing device 10D, the same components as those of the catheter fixing device 10 shown in FIG. 1 are denoted by the same reference numerals, and detailed description thereof is omitted.

As shown in FIGS. 11A and 11B, the catheter fixing device 10D according to the present embodiment includes a skin fixation part 12D, a hub fixation part 14D, and a movement mechanism 16D. As shown in FIG. 11B, the skin fixation part 12D has a frame portion 70 formed on an upper portion thereof. A housing concave portion 72 is formed in a part surrounded by the frame portion 70. A flexible member 74 is attached to the housing concave portion 72. The flexible member 74 is made of an elastic material such as urethane resin, rubber, or high molecular gel (for example, PVA and PVC). As shown in FIGS. 12A and 12B, the flexible member 74 protrudes above the frame portion 70 of the skin fixation part 12D. A bottom portion of the flexible member 74 adheres to the skin fixation part 12D, an upper portion of the flexible member 74 adheres to a bottom surface of the hub fixation part 14D, and the flexible member 74 is sandwiched between the skin fixation part 12D and the hub fixation part 14D and interlocks the skin fixation part 12D with the hub fixation part 14D to be movable relative to each other. That is, the movement mechanism 16D according to the present embodiment includes the flexible member 74.

According to the catheter fixing device 10D of the present embodiment, it is possible to configure, with a simple structure, the movement mechanism 16D that interlocks the hub fixation part 14D with the skin fixation part 12D to be movable relative to each other.

The catheter fixing devices 10, 10A, 10B, 10C, and 10D described in the above embodiments achieve the following effects.

The catheter fixing device 10 according to the present embodiment includes the skin fixation part 12 configured to come into contact with the skin 106, the hub fixation part 14 configured to retain the hub 102, and the movement mechanism 16 provided between the skin fixation part 12 and the hub fixation part 14 and interlocking the hub fixation part 14 with the skin fixation part 12 to be movable relative to each other.

According to the catheter fixing device 10 described above, even when body movement occurs, it is possible to prevent a distal end of the catheter 100 from coming off a puncture position by the hub fixation part 14 moving relative to the skin fixation part 12.

In the catheter fixing device 10, the movement mechanism 16 may interlock the hub fixation part 14 with the skin fixation part 12 to be movable relative to the skin fixation part 12 in an extending direction of the hub 102. According to this configuration, it is possible to prevent the catheter 100 from moving in the extending direction of the hub 102 due to detachment from the puncture position.

In the catheter fixing device 10 described above, the movement mechanism 16 may interlock the hub fixation part 14 with the skin fixation part 12 to be movable relative to each other in any direction of fixing surface directions of the skin fixation part 12. According to this configuration, a degree of freedom in the movement direction of the hub fixation part 14 is increased, and a position of the hub fixation part 14 is more appropriately adjusted with respect to movement of the skin 106 caused by the body movement.

In the catheter fixing device 10 described above, the movement mechanism 16 may include a linear movement member. The linear movement member includes a guide mechanism and a bearing, and the hub fixation part 14 can move relative to the skin fixation part 12 with a small resistance, and therefore the catheter 100 can be more effectively prevented from detaching.

In the catheter fixing device 10 described above, the linear movement member may be the free ball bearing 24. According to this configuration, the hub fixation part 14 can be smoothly moved relative to the skin fixation part 12 in the fixing surface directions.

In the catheter fixing device 10 described above, the linear movement member may be a linear guide. According to this configuration, the hub fixation part 14 can be smoothly displaced to prevent the catheter 100 from detaching.

In the catheter fixing device 10 described above, the linear movement member may include a guide member and the wheels 66 guided by the guide member. According to this configuration, the hub fixation part 14 can be smoothly displaced to prevent the catheter 100 from detaching.

In the catheter fixing device 10 described above, the movement mechanism 16 may be the flexible member 74 sandwiched in layers between the hub fixation part 14 and the skin fixation part 12. According to this configuration, the structure can be simplified, and a manufacturing cost can be reduced.

In the catheter fixing device 10 described above, the movement mechanism 16 may include a restriction member (for example, the stoppers 34) that restricts a movable region of the hub fixation part 14. According to this configuration, it is possible to prevent the hub fixation part 14 from falling off the movement mechanism 16.

The catheter fixing device 10 described above may include the lock mechanism 43 that fixes a position of the hub fixation part 14 with respect to the skin fixation part 12 at a predetermined position in a releasable manner. According to this configuration, because the movement of the hub fixation part 14 can be prevented when the catheter 100 is attached to the catheter fixing device 10, an attachment work of the catheter 100 can be easily performed.

In the catheter fixing device 10 described above, the skin fixation part 12 may include the adhesive portion 18 including an adhesive material layer on the fixing surface 12a that faces the skin 106. According to this configuration, the skin fixation part 12 can be fixed to the skin 106 via the adhesive portion 18.

In the catheter fixing device 10 described above, the hub fixation part 14 may include a protrusion portion (for example, the first retaining protrusion 46 and the second retaining protrusion 48) that fits and retains the hub 102 and a lock connector interlocked with the hub 102. According to this configuration, the hub 102 can be reliably fixed to the hub fixation part 14.

Although preferred embodiments according to the invention have been described above, the invention is not limited to the above-described embodiments, and it is needless to say that various modifications can be made without departing from the spirit of the invention.

What is claimed is:

1. A catheter fixing device comprising:
a skin fixation part configured to come into contact with skin;
a hub fixation part configured to retain a catheter hub; and
a movement mechanism provided between the skin fixation part and the hub fixation part and interlocking the hub fixation part with the skin fixation part such that the hub fixation part and the skin fixation part are movable relative to each other.

2. The catheter fixing device according to claim 1, wherein:
the movement mechanism interlocks the hub fixation part with the skin fixation part to be movable relative to the skin fixation part in an extending direction of the catheter hub.

3. The catheter fixing device according to claim 1, wherein:
the movement mechanism interlocks the hub fixation part with the skin fixation part such that the hub fixation part and the skin fixation part are movable relative to each other in any direction parallel to a fixing surface of the skin fixation part.

4. The catheter fixing device according to claim 1, wherein:
the movement mechanism comprises a linear movement member.

5. The catheter fixing device according to claim 4, wherein:
the linear movement member is a free ball bearing.

6. The catheter fixing device according to claim 4, wherein:
the linear movement member is a linear guide.

7. The catheter fixing device according to claim 4, wherein:
the linear movement member comprises a guide member and a wheel guided by the guide member.

8. The catheter fixing device according to claim 1, wherein:
the movement mechanism comprises a flexible member sandwiched between the hub fixation part and the skin fixation part.

9. The catheter fixing device according to claim 1, wherein:
the movement mechanism comprises a restriction member that restricts a movable region of the hub fixation part.

10. The catheter fixing device according to claim 1, further comprising:
a lock mechanism configured to fix a position of the hub fixation part with respect to the skin fixation part at a predetermined position in a releasable manner.

11. The catheter fixing device according to claim 1, wherein:
the skin fixation part comprises an adhesive material layer on a fixing surface that faces the skin.

12. The catheter fixing device according to claim 1, wherein:
the hub fixation part comprises a protrusion portion that fits and retains the catheter hub.

13. A catheter fixing device comprising:
a skin fixation part configured to come into contact with skin, the skin fixation part comprising:
an adhesive material layer on a fixing surface that faces the skin,
a pair of guide rails that extend in a length direction of the skin fixation part, the pair of guide rails defining a guide groove, and
a free ball bearing located at a bottom portion of the guide groove
a hub fixation part comprising:
a slider part on a lower surface side of the hub fixation part and located in the guide groove so as to be slidably supported by the free ball bearing, and
a retaining protrusion on a top surface side of the hub fixation part, the retaining protrusion being configured to retain the catheter hub.

14. A catheter fixing device comprising:
a skin fixation part configured to come into contact with skin, the skin fixation part comprising:
an adhesive material layer on a fixing surface that faces the skin, and
a pair of guide grooves located on opposite lateral sides of the skin fixation part in a width direction;
a hub fixation part comprising:
a top plate portion,
a retaining protrusion on a top surface side of the top plate portion, the retaining protrusion being configured to retain the catheter hub, and
a pair of side wall parts extending downward from opposite lateral sides of the top plate portion so as to cover the opposite lateral sides of the skin fixation part,
wherein each side wall part comprises a retaining part that protrudes into a respective one of the guide grooves; and
a plurality of balls located between each retaining part and a surface defining the respective guide groove.
